(19) United States
(12) Patent Application Publication
(10) Pub. No.: US 2017/0086961 A1
(43) Pub. Date: Mar. 30, 2017

(54) STRUCTURALLY ENHANCED TISSUE GRAFT

(71) Applicant: Vivex Biomedical, Inc., Marietta, GA (US)

(72) Inventor: Timothy Ganey, Tampa, FL (US)

(73) Assignee: VIVEX BIOMEDICAL, INC., Marietta, GA (US)

(21) Appl. No.: 14/865,798

(22) Filed: Sep. 25, 2015

(51) Int. Cl. A61F 2/02; B32B 9/02; A61L 27/38; B32B 37/16; A61L 27/36; B32B 3/28; B32B 37/00

(52) U.S. Cl. CPC A61F 2/02; B32B 3/28; B32B 9/02; B32B 37/0076; B32B 37/16; A61L 27/3604; A61L 27/3691; A61L 27/3804; A61F 2240/001; B32B 2535/00; B32B 2317/00; A61L 2430/40

(57) ABSTRACT

A structurally enhanced tissue graft has at least one placental membrane having a corrugated portion made of a series of pleated peaks and valleys. The structurally enhanced tissue graft further may have one or both of a first and second flat portion tissue membrane adhered to the corrugated portion. The first flat portion is adhered to an external surface along either peaks or valleys of the corrugated portion. The second flat portion tissue membrane adhered to the corrugated external surface along the corrugated portion opposing peaks or valleys. The first flat portion forms a top or bottom of the structurally enhanced tissue graft and the second flat portion forms an opposing bottom or top with the corrugated portion sandwiched therebetween.

### TECHNICAL FIELD

The present invention relates to a structurally enhanced placental tissue.

### BACKGROUND OF THE INVENTION

The use of tissue membranes for wound healing and tissue defect repair has been a well-accepted medical procedure for years. In U.S. Pat. No. 8,323,701B1; issued Dec. 12, 2012 entitled “Placental Tissue Graft” and assigned to Mimedx Group, Inc. they reported the use of human placental membrane (e.g. amniotic membrane or tissue) has been used for various types of reconstructive surgical procedures since the early 1900's. In that patent, Daniels et al received a grant on a specific structure defined by claim 1:

“1. A tissue graft consisting of:
a first membrane comprising modified amnion wherein the modified amnion has a first side which is an exposed basement membrane and a second side which is an exposed jelly-like fibroblast cellular layer; and
one or more additional membranes sequentially layered such that the first additional membrane is layered adjacent to the exposed fibroblast layer of the first membrane,
wherein the at least one or more additional membranes is selected from the group consisting of amnion, chorion, allograft pericardium, allograft acellular dermis, amniotic membrane, Wharton's jelly, and combinations thereof.”

The laminate tissue graft had a thickness of 0.2 mm to 10 mm when additional layers numbering 1 to 10 were laminated to the first membrane.

The need to laminate is clear when a single dehydrated placental tissue membrane has a thickness of 0.1 mm. The tissue graft is very thin and flexible, so much so, great care must be used to keep the membrane from wrinkling with the sticky exposed jelly-like fibroblast cellular layer adhering to itself rendering the tissue graft useless.

The main drawback of laminating flat membranes is the lack of tissue contacting surface area compared to the total square area of the individual membranes. A two layer laminate has a tissue contact area of 50% of the individual membranes and a ten layer laminate is 10% of the individual membranes. This renders the tissue graft very expensive simply to achieve the desired stiffness for handling.

The present invention employs as few as one single membrane in a unique configuration that maximizes the surface contact area with the wound to be treated while dramatically stiffening the tissue graft.

### SUMMARY OF THE INVENTION

A structurally enhanced tissue graft has at least one placental membrane having a corrugated portion made of a series of pleated peaks and valleys. The structurally enhanced tissue graft further may have one or both of a first and second flat portion tissue membrane adhered to the corrugated portion. The first flat portion is adhered to an external surface along either peaks or valleys of the corrugated portion. The second flat portion tissue membrane adhered to the corrugated external surface along the corrugated portion opposing peaks or valleys. The first flat portion forms a top or bottom of the structurally enhanced tissue graft and the second flat portion forms an opposing bottom or top with the corrugated portion sandwiched therebetween.

In one embodiment, the first flat portion extends laterally from a side of the corrugated portion and is part of the placental tissue membrane and the first flat portion is folded onto above or below the corrugated portion to form the respective top or bottom of the structurally enhanced tissue graft. Similarly, the second flat portion extends from an opposing side of the corrugated portion and the second flat portion is folded onto above or below the corrugated portion to form the respective top or bottom of the structurally enhanced membrane.

The at least one placental membrane has a membrane thickness of 0.1 mm when formed from a single membrane layer. The pleated peaks or valleys have a height of 3 to 100 times the thickness of the placental tissue membrane. The placental tissue membrane can be dried on a mandrel or die to form the pleated peaks and valleys. The structurally enhanced tissue graft preferably has been freeze dried. The total height of the structurally enhanced tissue graft is in the range of 1.0 mm to 10.0 mm. The external surfaces are an epithelial surface which an exposed jelly-like fibroblast cellular layer. The first or second flat portions can be selected from the group consisting of amnion, chorion, allograft pericardium, allograft acellular dermis, amniotic membrane, Wharton's jelly, and combinations thereof.

At ends of the pleated peaks and valleys and the first or second flat portions form open troughs or channels for infusion of blood flow during implantation. The open troughs are straight, curved or wavy. The first flat layer or second flat layer can have holes or perforations. The structurally enhanced tissue graft can be made using a plurality of placental tissue graft membranes that are laminated together to form the corrugated portion.

The invention may include a method of making a structurally enhanced tissue graft comprises the steps of processing a placental tissue; cutting the tissue to sizes; drying the cut tissue by placing on a die or mandrel to form a corrugated tissue having pleated peaks and valleys; removing the dried tissue from the die or mandrel; and adding or folding a flat top and or bottom onto the corrugated tissues and adhering to form a structurally enhanced tissue graft as an assembly.

The method of making a structurally enhanced tissue graft further may comprise the step of freeze-drying the structurally enhanced tissue graft.

### BRIEF DESCRIPTION OF THE DRAWINGS

The invention will be described by way of example and with reference to the accompanying drawings in which:

FIG. 1 is an end or plan view of a placental tissue membrane.

FIG. 2 is an end view of a placental tissue membrane of a first embodiment of the present invention.

FIG. 2A is a schematic view with arrows showing the direction that the first flat portion can be folded.

FIG. 2B shows the first flat portion of FIG. 2A moved over the corrugated portion and adhered to the peaks as shown.

FIG. 3 is a second embodiment having a first and a second flat portion with the corrugated portion positioned therebetween.

FIG. 3A shows schematically with direction arrows how the flat portions can be folded over and under the corrugated portion to form the second embodiment.

FIG. 3B illustrates the finished second embodiment with the first flat portion adhered to the peaks and the second flat portion adhered to the valleys.

FIG. 4 shows a top view of the corrugated portion of a mandrel or drying die formed with a plurality of straight peaks and valleys.

FIG. 5 shows a top view of the corrugated portion of a mandrel or drying die with a plurality of wavy peaks and valleys.

FIG. 6 is an exemplary view of various profiles of the peaks and valleys can take.

FIG. 7 is a perspective view of the first embodiment made as two separate pieces adhered together.

FIG. 8 is the second embodiment made as three separate pieces adhered together.

FIG. 9 shows a combination of a first embodiment adhered to a second embodiment to form a double wall stack.

FIG. 10 is a triple wall stack.

FIG. 11 shows a dried placental tissue membrane.

### DETAILED DESCRIPTION OF THE INVENTION

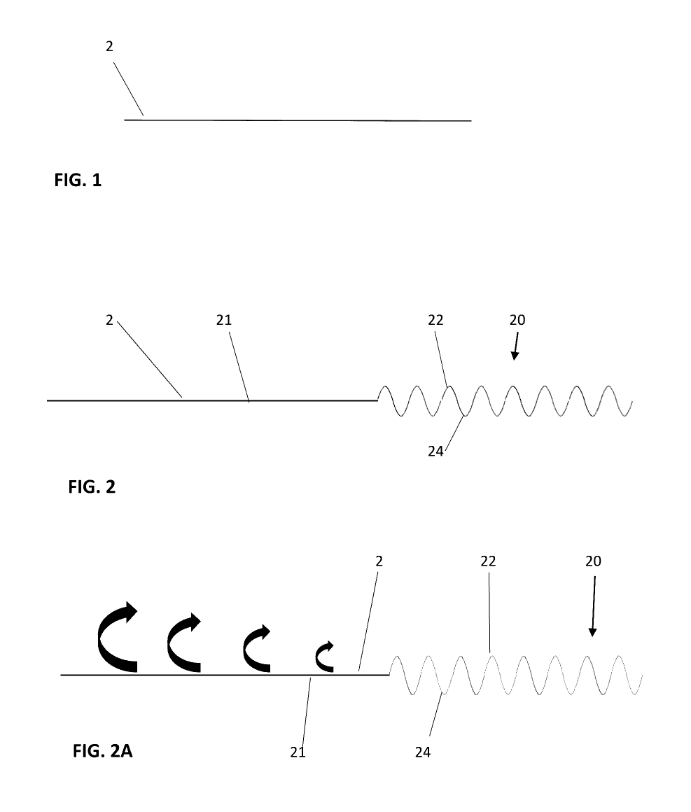

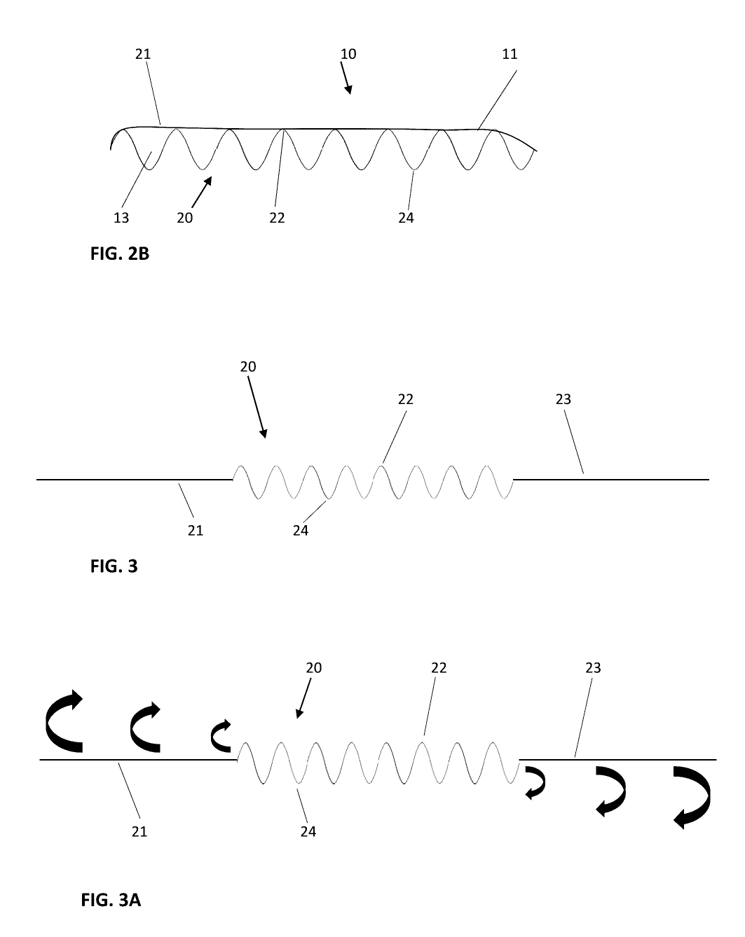

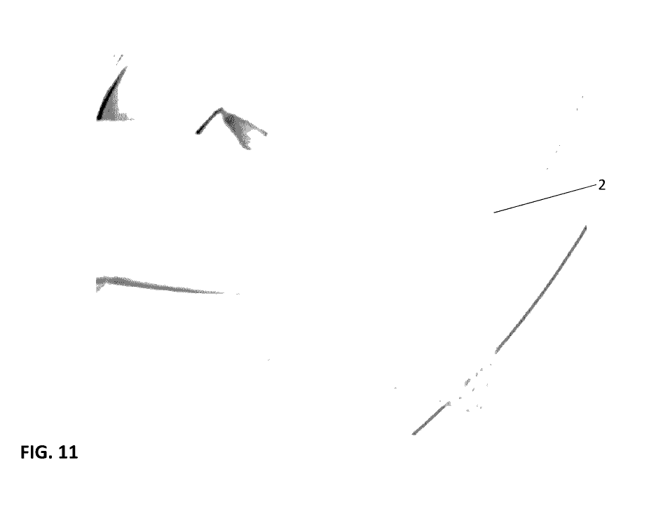

With reference to FIGS. 1-3B, various constructions of the present invention are illustrated. FIG. 1 shows the placental tissue membrane 2 from and end view. This is best shown in the photo of FIG. 11 showing it formed as a flat or straight square or rectangular membrane 2. FIG. 2 is an end view showing the membrane 2 when dried on a mandrel or die having at least one lateral pleated portion 20 that is formed as corrugated peaks 22 and valleys 24. FIG. 2A illustrates that the flat portion 21 of the membrane 2 when directionally folded over can form a single sheet corrugate structure 11 made of the placental tissue membrane 2 as illustrated in FIG. 2 where the flat portion 21 lays on the peaks 22 or top of the pleated portion 20 of the placental membrane 2 in such a fashion that the placental membrane 2 in the regions contacted along the peaks 22 will adhere to the flat portion 21 making a structurally enhanced placental tissue 10. As can be appreciated in referring to FIG. 2, when the placental membrane 2 as a flat plate or sheet is corrugated, a large increase in surface area is created when compared to flat laminated sheets. This increase in surface area allows more placental tissue to be positioned into contact with the wound or area of a patient to be treated. Additionally, by having the membrane 2 folded onto itself, these pleated wave patterns of the corrugation structure 11 can be maintained during the healing process. As illustrated in FIG. 2B, the enhanced structural strength placental tissue 10 forms open troughs or channels 13 that are defined by the area enveloped by the flat portion 21 and the valleys 24 of the corrugated structure 11. These troughs or channels 13 extend across the length of the membrane 2 and are open at both ends. This enables other fluids of the patient to flow into enhancing the amount of placental tissue in contact with the patient's bodily fluids. In this case, when immersed in blood, the beneficial healing capability of the placental tissue is enhanced by this dramatic increase in surface area in contact with the wound area.

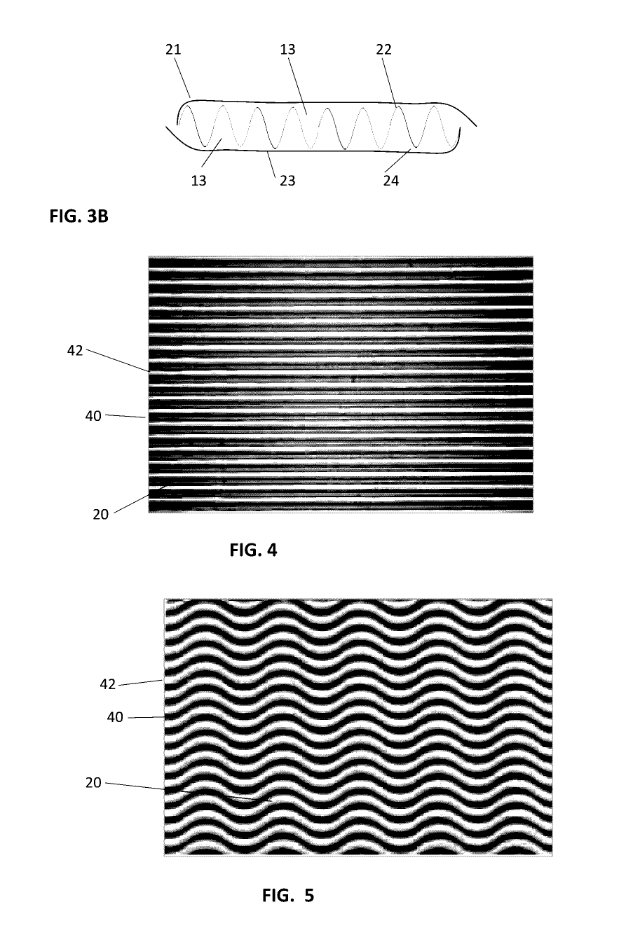

With reference to FIG. 3, a second embodiment is illustrated wherein each lateral side 21, 23 of the tissue membrane 2 is shown as a flat and interposed in between the two flat portions 21, 23 on the lateral extremes is the corrugated portion 20 having the peaks 22 and valleys 24. As illustrated in FIG. 3A, when the left hand lateral portion 21 is folded upwardly and the right hand lateral portion 23 is folded downwardly, the peaks 22 are contracted by the left hand lateral portion 21 and adhered directly thereto and the valleys 24 or bottom are adhered to the right hand lateral portion 23. When this occurs, both the top and the bottom at the extremes of the peaks 22 and valleys 24 are in direct contact with one of the flat portions 21 or 23. This makes a corrugated enhanced structural strength placental tissue membrane 10 of substantially increased strength.

With reference to FIGS. 4 and 5, mandrel dies 40, 50 are illustrated. These dies 40, 50 have the peaks 22 and valleys 24 cut into them. The placental tissue corrugated portion 20 is laid on these dies 40, 50 with the lateral portions 21, 23 laying on a flat surface. As the tissue membrane 2 dries, the shape of the corrugations 42, 52 is imparted on the membrane 2 and fixed thereto in such a fashion that the corrugated portion 20 of the membrane 2 will have the structurally enhanced feature as previously discussed in FIGS. 1-3B.

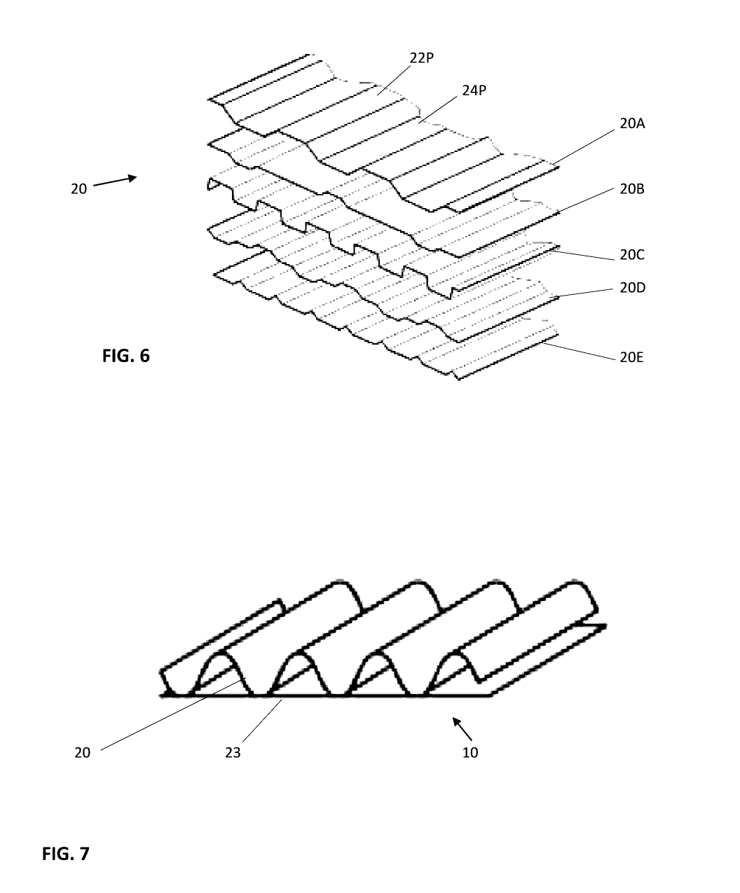

With reference to FIG. 6, the peaks 22 and valleys 24 can be made in a number of exemplary shapes. The corrugated portions 20, as shown, can be made wherein the peaks 22 are formed as a flattened plateau 22P and the valleys 24 have a lower flat portion 24P as well. This is illustrated in each of the top four examples of corrugated portions 20A, 20B, 20C and 20D illustrated in FIG. 6. The lower embodiment 20E has the peaks 22 spaced widely with more lateral portions. Therefore the amplitude of the peaks 22 as illustrated occur and there are no valleys 24 as illustrated in the first two embodiments of FIGS. 1-3B, only peaks 22. Any of these structures can be used to enhance the structurally integrity of the tissue membrane 2 by allowing the lateral portions 21, 23 to fold over as previously discussed. What happens with the trapezoidal type peak 22 and valley 24 is that a larger surface are is maintained for adhering the folded over flat portions 21, 23 to the corrugated portion 20A-20D and even 20E as can be appreciated from the views of FIG. 6.

FIG. 7, as shown, is made as a two piece structurally enhanced tissue graft wherein the pleated corrugated portion 20 is one piece and the top layer 21 is adhered to and is a separate piece relative to the corrugated portion 20.

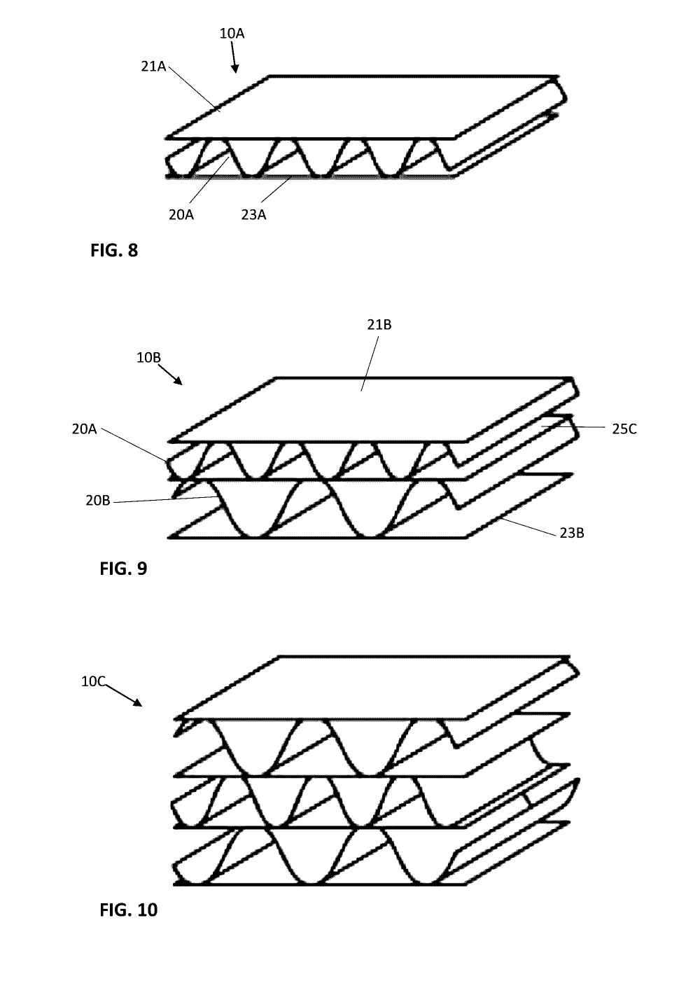

With reference to FIG. 8, a perspective view of a three piece structure 10A is illustrated. In this third embodiment 10, the entire middle layer is the corrugated portion 20A and a top layer 21A and bottom layer 23A that are separate pieces that are formed as an assembly. These top and bottom layers 21A, 23A do not need to be integral with the corrugated portion 20A and could be made of separate plates as shown. These embodiments can be then made as illustrated in FIG. 8 as a single structurally enhanced placental tissue 10A or can be stacked as illustrated in FIG. 9 having two corrugations 20A, 20B with a flat layer 25C placed in between and a flat top 21B and bottom layer 23B as illustrated. When this is done, the height of the structurally enhanced placental tissue 10B is increased dramatically. As shown the lower portion does not need to be the same size having larger peaks and valleys with a smaller repeating sequence in this fashion this entire shape of the structures when stacked together can be varied as the end user desires.

With reference to FIG. 10, a triple stack 10C is shown wherein two large amplitudes are shown on the top and bottom and the middle has smaller peaks and valleys. This creates a triple stack and further increases the height. These variations of the structure can all be used with the present invention. Whether the lateral portions 21, 23 are integral to the corrugation 20 or whether they are provided as separate pieces, in any event, the entire membrane structure 10, 10A-10C has a large increase in surface area to provide additional healing properties of the placental tissue.

FIG. 11 shows a placental tissue 2 when produced as a flat plane as illustrated in FIG. 1. It is this very tissue that can be laid on a mandrel 40 or die 50 when wet and dried that can achieve these shapes shown in FIGS. 1-10.

As illustrated, the placental tissue 2 can be one of an amnion layer, a chorion layer or a combination of amnion and chorion layers. The thickness of such a single layer membrane is extremely thin, by providing a single layer amniotic tissue into a corrugated material increases the structural integrity. When the membrane 2 is corrugated without a lateral top or bottom folded over or a separate top or bottom adhered to the peaks and valleys, the pleated corrugated portion 20 membrane 2 itself will have an increased stiffness in one direction however it can be bent easily and folded. This is even true of the embodiment of FIG. 2B wherein only a top 21 or bottom 23 layer is provided attached to the corrugated portions 20. When this occurs, as can easily be appreciated, the entire assembly can be folded on itself or formed into a cylindrical tube if so desired or any other shape. However, bending in a longitudinal direction along the length of the troughs of the peaks and valleys is quite difficult whether or not only one of an upper or lower layer is employed. It is important to understand that by increasing the surface area of the placental tissue, not only enhances the structural strength, but increases the surface area in contact with the wound and provides for improved healing when made as a single layer.

Another variation of the present invention is that the single placental tissue layer can be made as a laminate structure having 1-10 additional layers of amniotic tissue laminated together. This would be similar to the prior art disclosed U.S. Pat. No. 8,323,701 in the background of the invention. If the tissue is produced as a multiple laminated structure having one base member and several laminated sheets, it can still be dried and formed with corrugations and achieve the benefits of the present invention as an alternative to a single membrane. Therefore, this invention is to include not only a single membrane of placental tissue, but any combination of layers whether they be laminated together or otherwise adhered during the drying process to achieve a structurally enhanced corrugation of the enhanced structural placental tissue membrane.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

1. A freeze-dried structurally enhanced placental tissue graft made from placental tissue comprises:
at least one placental tissue membrane formed from a single membrane having a corrugated portion of placental tissue made of a series of pleated peaks and valleys and a lateral flat portion of placental tissue adhered to the corrugated portion to form the structurally enhanced placental tissue graft upon drying, wherein the flat portion when placed in contact to an outer surface along either peaks or valleys of the corrugated portion adheres directly to the corrugated portion wherein the flat portion in combination with the corrugated portion form open channels configured for infusion of blood flow during implantation and the combination of the folded over lateral flat portion and the corrugated portion have the same exposed external surfaces and the channels created by the folded flat portion and the corrugated portion have the same internal surfaces; wherein the exposed external surfaces are an epithelial surface with an exposed jelly-like fibroblast cellular layer; and
wherein the structurally enhanced tissue graft has been freeze dried.

2. (canceled)

3. The structurally enhanced tissue graft of claim 2 further comprises a second flat portion tissue membrane adhered to the corrugated external surface along the corrugated portion opposing peaks or valleys, the first flat portion forming a top or bottom of the structurally enhanced tissue graft and the second flat portion forming an opposing bottom or top with the corrugated portion sandwiched therebetween.

4. The structurally enhanced tissue graft of claim 2 wherein the first flat portion extends laterally from a side of the corrugated portion and is part of the placental tissue membrane and the first flat portion is folded onto above or below the corrugated portion to form the respective top or bottom of the structurally enhanced tissue graft.

5. The structurally enhanced tissue graft of claim 3 wherein the first flat portion extends laterally from a side of the corrugated portion and is part of the placental tissue membrane and the first flat portion is folded onto above or below the corrugated portion to form the respective top or bottom of the structurally enhanced tissue graft and the second flat portion extends from an opposing side of the corrugated portion and the second flat portion is folded onto above or below the corrugated portion to form the respective top or bottom of the structurally enhanced membrane.

6. The structurally enhanced tissue graft of claim 1 wherein the at least one placental membrane has a membrane thickness of 0.1 mm when formed from a single membrane layer.

7. The structurally enhanced tissue graft of claim 1 wherein the pleated peaks or valleys have a height of 3 to 100 times the thickness of the placental tissue membrane.

8. The structurally enhanced tissue graft of claim 1 wherein the at least one placental tissue membrane is dried on a mandrel or die to form the pleated peaks and valleys.

9. (canceled)

10. The structurally enhanced tissue graft of claim 1 wherein the total height of the structurally enhanced tissue graft is 1.0 mm to 10.0 mm.

11. (canceled)

12. The structurally enhanced tissue graft of claim 3 wherein the first or second flat portions are selected from the group consisting of amnion, chorion, allograft pericardium, allograft acellular dermis, amniotic membrane, Wharton's jelly, and combinations thereof.

13. The structurally enhanced tissue graft of claim 12 wherein at ends of the pleated peaks and valleys and the first or second flat portions form open troughs or channels for infusion of blood flow during implantation.

14. The structurally enhanced tissue graft of claim 13 wherein the open troughs are straight, curved or wavy.

15. The structurally enhanced tissue graft of claim 3 wherein the first flat layer or second flat layer has holes.

16. The structurally enhanced tissue graft of claim 1 wherein a plurality of placental tissue graft membranes are laminated together to form the corrugated portion.

17. A method of making a structurally enhanced tissue graft comprises the steps of:
processing a placental tissue;
cutting the tissue to sizes;
drying the cut tissue by placing on a die or mandrel to form a corrugated tissue having pleated peaks and valleys;
removing the dried tissue from the die or mandrel; and
adding or folding a flat top and or bottom onto the corrugated tissues and adhering to form a structurally enhanced tissue graft as an assembly.

18. The method of making a structurally enhanced tissue graft of claim 17 further comprises the step of freeze-drying the structurally enhanced tissue graft.
